(19) United States
(12) Patent Application Publication
(10) Pub. No.: US 2015/0272979 A1
(43) Pub. Date: Oct. 1, 2015

(54) COMBINATION THERAPEUTIC COMPOSITION

(71) Applicant: Hoffman La-Roche Inc., Nutley, NJ (US)

(72) Inventors: Patrick F. Smith, Montville, NJ (US); Tom W. Chu, Montclair, NJ (US); Janet Hammond, Montclair, NJ (US)

(21) Appl. No.: 14/679,779

(22) Filed: Apr. 6, 2015

Related U.S. Application Data
(63) Continuation of application No. 13/915,013, filed on Jun. 11, 2013, now abandoned.
(60) Provisional application No. 61/658,740, filed on Jun. 12, 2012.

(51) Int. Cl. A61K 31/7068; A61K 31/549; A61K 31/407

(52) U.S. Cl. CPC A61K 31/7068; A61K 31/407; A61K 31/549

(57) ABSTRACT

Embodiments disclosed in the present application relate to a composition that can include a hepatitis C viral nucleoside polymerase inhibitor, or pharmaceutically acceptable salt or prodrug thereof, a hepatitis C viral protease inhibitor, or pharmaceutically acceptable salt or prodrug thereof, and a hepatitis C viral non-nucleoside polymerase inhibitor, or pharmaceutically acceptable salt or prodrug thereof. Additional embodiments disclosed relate to methods for treating a disease condition such as a hepatitis C virus infection, liver fibrosis and/or impaired liver function with a hepatitis C viral nucleoside polymerase inhibitor, or pharmaceutically acceptable salt or prodrug thereof, a hepatitis C viral protease inhibitor, or pharmaceutically acceptable salt or prodrug thereof, and a hepatitis C viral non-nucleoside polymerase inhibitor, or pharmaceutically acceptable salt or prodrug thereof.

### CROSS-REFERENCE TO RELATED PATENT APPLICATIONS

The present application is a continuation of U.S. application Ser. No. 13/915,013 filed Jun. 11, 2013 which claims the benefit of U.S. Provisional Patent Application No. 61/658,740, filed Jun. 12, 2012. The content of both applications are incorporated herein by reference in their entirety.

### FIELD

The present application relates to compositions and methods for the treatment of a disease condition such as a hepatitis C virus infection, liver fibrosis, and impaired liver function.

### BACKGROUND

Hepatitis C virus (HCV) infection is the most common chronic blood borne infection in the United States. Although the numbers of new infections have declined, the burden of chronic infection is substantial, with Centers for Disease Control estimates of 3.9 million (1.8%) infected persons in the United States. Chronic liver disease is the tenth leading cause of death among adults in the United States, and accounts for approximately 25,000 deaths annually, or approximately 1% of all deaths. Studies indicate that 40% of chronic liver disease is HCV-related, resulting in an estimated 8,000-10,000 deaths each year. HCV-associated end-stage liver disease is the most frequent indication for liver transplantation among adults.

Antiviral therapy of chronic hepatitis C has evolved rapidly over the last decade, with significant improvements seen in the efficacy of treatment. Nevertheless, even with using the standard of care (SOC) combination therapy of pegylated IFN-α, plus ribavirin, 40% to 50% of patients fail therapy, i.e., are nonresponders or relapsers. These patients currently have no effective therapeutic alternative. In particular, patients who have advanced fibrosis or cirrhosis on liver biopsy are at significant risk of developing complications of advanced liver disease, including ascites, jaundice, variceal bleeding, encephalopathy, and progressive liver failure, as well as a markedly increased risk of hepatocellular carcinoma.

The high prevalence of chronic HCV infection has important public health implications for the future burden of chronic liver disease in the United States. Data derived from the National Health and Nutrition Examination Survey (NHANES III) indicate that a large increase in the rate of new HCV infections occurred from the late 1960s to the early 1980s, particularly among persons between 20 to 40 years of age. It is estimated that the number of persons with long-standing HCV infection of 20 years or longer could more than quadruple from 1990 to 2015, from 750,000 to over 3 million. The proportional increase in persons infected for 30 or 40 years would be even greater. Since the risk of HCV-related chronic liver disease is related to the duration of infection, with the risk of cirrhosis progressively increasing for persons infected for longer than 20 years, a substantial increase in cirrhosis-related morbidity and mortality is likely to result among patients infected between the years of 1965-1985.

HCV is an enveloped positive strand RNA virus in the Flaviviridae family. The single strand HCV RNA genome is approximately 9500 nucleotides in length and has a single open reading frame (ORF) encoding a single large polyprotein of about 3000 amino acids. In infected cells, this polyprotein is cleaved at multiple sites by cellular and viral proteases to produce the structural and non-structural (NS) proteins of the virus (NS2, NS3, NS4, NS4A, NS4B, NSSA, and NSSB).

### SUMMARY

One aspect of the invention relates to a composition that comprises a first compound, or a pharmaceutically acceptable salt or prodrug thereof, wherein the first compound is

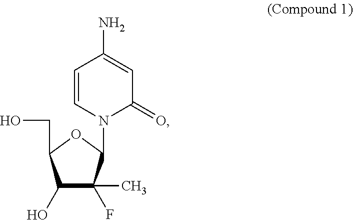

a second compound, or a pharmaceutically acceptable salt or prodrug thereof, wherein the second compound is

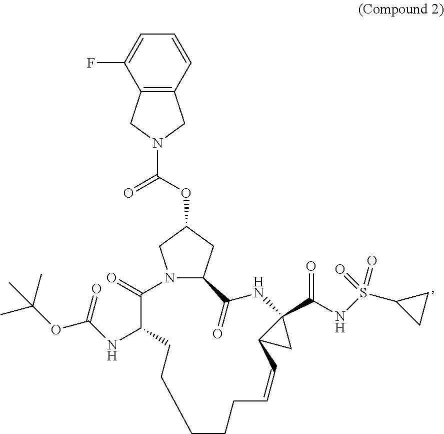 [SMILES: CC(C)(C)OC(=O)N[C@H]1CCCCC/C=C\[C@@H]2C[C@@]2(C(=O)NS(=O)(=O)C2CC2)NC(=O)[C@@H]2C[C@@H](OC(=O)N3Cc4cccc(F)c4C3)CN2C1=O]

and a third compound, or a pharmaceutically acceptable salt or prodrug thereof, wherein the third compound is

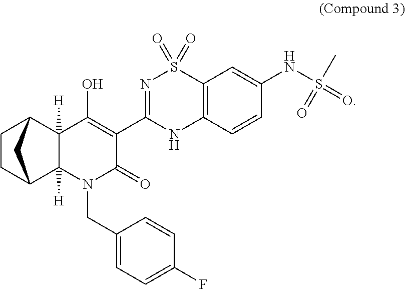

In another aspect the invention relates to a composition comprising Compound 1, Compound 2, and Compound 3, or pharmaceutically acceptable salts or prodrugs thereof, wherein the composition additionally comprises one or more therapeutic agents. In one embodiment the one or more therapeutic agents are ribavirin and ritonavir.

In one embodiment of the invention, the prodrug of the first compound can be the diisobutyl ester prodrug of β-D-2′-deoxy-2′-fluoro-2′-C-methylcytidine

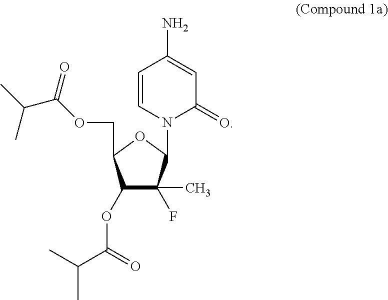

In another embodiment, the salt of the second compound can be the sodium salt of 1S, 4R, 6S, 14S, 18R)-4-Fluoro-1,3-dihydro-isoindole-2-carboxylic acid 14-tert-butoxycarbonylamino-4-cyclopropanesulfonylaminocarbonyl-2,15-dioxo-3,16-diaza-tricyclo[14.3.0.04,6]nonadec-7-en-18-yl ester (Compound 2a).

In another aspect the invention relates to a composition comprising Compound 1a, Compound 2a, and Compound 3, or a pharmaceutically acceptable salt or prodrug of Compound 3, wherein the composition additionally comprises one or more therapeutic agents that are ribavirin and ritonavir.

In another aspect the invention relates to the use of such compositions for ameliorating or treating a disease condition in a patient population, and/or for the preparation of a medicament for ameliorating or treating such a disease condition. For example, the disease condition can be selected from a hepatitis C virus infection, liver fibrosis, and impaired liver function. In one embodiment the invention relates to the use of a composition comprising Compound 1, Compound 2, and Compound 3, or pharmaceutically acceptable salts or prodrugs thereof, for ameliorating or treating hepatitis C virus infection, liver fibrosis, and impaired liver function.

In another aspect the invention relates to a method for ameliorating or treating a disease condition in a patient population that comprises administering a therapeutically effective amount of a first compound, or a pharmaceutically acceptable salt or prodrug thereof, wherein the first compound is Compound 1; a therapeutically effective amount of a second compound, or a pharmaceutically acceptable salt or prodrug thereof, wherein the second compound is Compound 2; and a therapeutically effective amount of a third compound, or a pharmaceutically acceptable salt or prodrug thereof, wherein the third compound is Compound 3; to a subject suffering from the disease condition. In one embodiment, the disease condition can be selected from a hepatitis C virus infection, liver fibrosis, and impaired liver function.

In another aspect the invention relates to a use of Compound 1 or a pharmaceutically acceptable salt or prodrug thereof for ameliorating or treating a disease condition in a patient population, and/or for the preparation of a medicament for ameliorating or treating such a disease condition, wherein Compound 1 or a pharmaceutically acceptable salt or prodrug thereof is manufactured for use in combination with Compound 2 or a pharmaceutically acceptable salt or prodrug thereof; and wherein Compound 1 and Compound 2 or pharmaceutically acceptable salts or prodrugs thereof are manufactured for use in combination with Compound 3 or a pharmaceutically acceptable salt or prodrug thereof; wherein the disease condition is selected from hepatitis C virus infection, liver fibrosis, and impaired liver function.

In one embodiment of the invention the method or use for ameliorating or treating a disease condition in a patient population comprises administering one or more additional therapeutic agents. In another embodiment the one or more additional therapeutic agents are ribavirin and ritonavir.

In another aspect the invention relates to a method for ameliorating or treating a disease condition in a patient population that comprises administering a therapeutically effective amount of Compound 1a, Compound 2a, Compound 3, or a pharmaceutically acceptable salt or prodrug of Compound 3, and additional therapeutic agents that are ribavirin and ritonavir, to a subject suffering from the disease condition. In one embodiment, the disease condition can be selected from a hepatitis C virus infection, liver fibrosis, and impaired liver function.

These and other embodiments are described in greater detail below.

### BRIEF DESCRIPTION OF THE DRAWINGS

FIG. 1 shows a pictorial representation of five treatment regimes using the compounds shown.

### DETAILED DESCRIPTION

Embodiments include, but are not limited to, therapeutic compositions and their use in the treatment and/or amelioration of a disease condition. In some embodiments, the disease condition can be selected from a hepatitis C virus infection, liver fibrosis, and/or impaired liver function.

Unless defined otherwise, all technical and scientific terms used herein have the same meaning as commonly understood by one of ordinary skill in the art to which the embodiments belong. All publications mentioned herein are incorporated herein by reference to disclose and describe the methods and/or materials in connection with which the publications are cited.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

Where a range of values is provided, it is understood that each intervening value, to the tenth of the unit of the lower limit unless the context clearly dictates otherwise, between the upper and lower limit of that range and any other stated or intervening value in that stated range is encompassed within the embodiments. The upper and lower limits of these smaller ranges, which may independently be included in the smaller ranges, are also encompassed within the invention, subject to any specifically excluded limit in the stated range. Where the stated range includes one or both of the limits, ranges excluding either or both of those included limits are also included in the embodiments.

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid, phosphoric acid and the like. Pharmaceutical salts can also be obtained by reacting a compound with an organic acid such as aliphatic or aromatic carboxylic or sulfonic acids, for example acetic, succinic, lactic, malic, tartaric, citric, ascorbic, nicotinic, methanesulfonic, ethanesulfonic, p-toluensulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine, lysine, and the like. The sodium salt of Compound 2 is a non-limiting example of a pharmaceutically acceptable salt.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. Compound 1a is a non-limiting example of a prodrug (in this case a prodrug of Compound 1). Conventional procedures for the selection and preparation of suitable prodrug derivatives are described, for example, in Design of Prodrugs, (ed. H. Bundgaard, Elsevier, 1985), which is hereby incorporated herein by reference for the purpose of describing procedures and preparation of suitable prodrug derivatives.

The term “effective amount” is used to indicate an amount of an active compound, or pharmaceutical agent, that elicits the biological or medicinal response indicated. For example, an effective amount of compound can be the amount needed to prevent, alleviate or ameliorate symptoms of disease or prolong the survival of the subject being treated. This response may occur in a tissue, system, animal or human and includes alleviation of the symptoms of the disease being treated. Determination of an effective amount is well within the capability of those skilled in the art, especially in light of the detailed disclosure provided herein. The effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize. In general, an effective amount of the compositions described herein, and optionally one or more additional antiviral agents, is an amount that is effective to reduce viral load or achieve a sustained viral response to therapy.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “host,” “subject,” and “patient” are used interchangeably herein, and refer to a mammal, including, but not limited to, murines, simians, humans, mammalian farm animals, mammalian sport animals, and mammalian pets.

As used herein, the term “hepatic fibrosis,” used interchangeably herein with “liver fibrosis,” refers to the growth of scar tissue in the liver that can occur in the context of a chronic hepatitis infection.

As used herein, the term “liver function” refers to a normal function of the liver, including, but not limited to, a synthetic function, including, but not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

The term “sustained viral response” (SVR; also referred to as a “sustained response” or a “durable response”), as used herein, refers to the response of an individual to a treatment regimen for HCV infection, in terms of serum HCV titer. For example, a “sustained viral response” refers to no detectable HCV RNA (e.g., less than about 500, less than about 200, or less than about 100 genome copies per milliliter serum) found in the patient's serum for a period of at least about one month (“SVR4”), at least about two months (“SVR8”), at least about three months (“SVR12”), at least about four months (“SVR16”), at least about five months (“SVR20”), and/or at least about six months (“SVR24”) following cessation of treatment.

The compound, β-D-2′-deoxy-2′-fluoro-2′-C-methylcytidine (Compound 1) has been demonstrated to be effective in inhibiting HCV replication. Although this invention is not limited by any particular theory, it is believed that Compound 1 inhibits HCV replication by inhibiting the HCV RNA polymerase, an enzyme involved in the replication of the hepatitis C virus. Compound 1 can be obtained using methods known to those skilled in the art, such as those methods described in U.S. Pat. No. 7,419,572, which is hereby incorporated by reference in its entirety. Pharmaceutically acceptable salts and prodrugs of Compound 1 can be utilized in the compositions described herein. For example, the diisobutyl ester prodrug of β-D-2′-deoxy-2′-fluoro-2′-C-methylcytidine

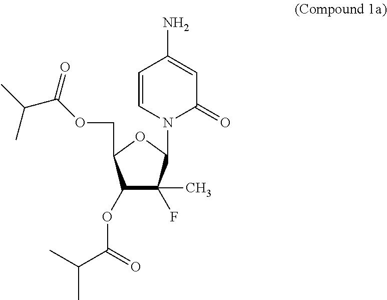

has been shown to have increased permeability that led to increased plasma exposure, and thereby improved anti-viral efficacy.

The compound, (1S,4R,6S,14S,18R)-4-Fluoro-1,3-dihydro-isoindole-2-carboxylic acid 14-tert-butoxycarbonylamino-4-cyclopropanesulfonylaminocarbonyl-2,15-dioxo-3,16-diaza-tricyclo[14.3.0.04,6]nonadec-7-en-18-yl ester (Compound 2) has shown to be effective in inhibiting HCV replication. The aforementioned compound can be obtained using methods known to those skilled in the art, including, for example, those methods disclosed in U.S. Pat. No. 7,491,794, which is hereby incorporated by reference in its entirety. Although this invention is not limited by any particular theory, Compound 2 is believed to inhibit the HCV protease, in particular the NS3/4A protease. Pharmaceutically acceptable salts and prodrugs of Compound 2 can be utilized in the compositions described herein. For example, the sodium salt of Compound 2 can be included in compositions described herein and is designated herein as Compound 2a. The structure and methods for producing Compound 2a are described in U.S. Publication No. 2007-0054842, filed on Jul. 21, 2006, which is hereby incorporated by reference in its entirety.

The compound, N-{3-R1R,2S,7R,8S)-3-(4-Fluoro-benzyl)-6-hydroxy-4-oxo-3-aza-tricyclo[6.2.1.0^{2,7}]undec-5-en-5-yl]-1,1-dioxo-1,4-dihydro-1λ⁶-benzo[1,2,4]thiadiazin-7-yl}-methanesulfonamide (Compound 3) has been demonstrated to be effective in inhibiting HCV replication. Compound 3 can be obtained using methods known to those skilled in the art, such as those methods described in U.S. Pat. No. 7,939,524, which is hereby incorporated by reference in its entirety. Although this invention is not limited by any particular theory, it is believed that Compound 3 is a non-nucleoside inhibitor of the HCV RNA polymerase, an enzyme involved in the replication of the hepatitis C virus.

For the compounds described herein, each stereogenic carbon can be of R or S configuration. Although the specific compounds exemplified in this application can be depicted in a particular configuration, compounds having either the opposite stereochemistry at any given chiral center or mixtures thereof are also envisioned unless otherwise specified. When chiral centers are found in the salts or prodrugs of the compounds, it is to be understood that the compounds encompasses all possible stereoisomers unless otherwise indicated. In addition it is understood that, in any compound described herein having one or more double bond(s) generating geometrical isomers that can be defined as E or Z, each double bond may independently be E or Z a mixture thereof. Likewise, all tautomeric forms are also intended to be included.

Some embodiments described herein relate to a composition that can include Compound 1, or a pharmaceutically acceptable salt or prodrug thereof; Compound 2, or a pharmaceutically acceptable salt or prodrug thereof; and Compound 3, or a pharmaceutically acceptable salt or prodrug thereof. In an embodiment, the prodrug of Compound 1 can be Compound 1a. In some embodiments, the salt of Compound 2 can be Compound 2a.

An embodiment described herein relates to a composition comprising Compound 1, or a pharmaceutically acceptable salt or prodrug thereof; Compound 2, or a pharmaceutically acceptable salt or prodrug thereof; and Compound 3, or a pharmaceutically acceptable salt or prodrug thereof. In some embodiments, the prodrug of Compound 1 can be Compound 1a. In an embodiment, the salt of Compound 2 can be the Compound 2a.

In some embodiments, the composition can further include a pharmaceutically acceptable excipient, diluent and/or carrier, such as those described herein.

Various amounts of Compound 1, or a pharmaceutically acceptable salt or prodrug thereof, can be included in the compositions described herein. In some embodiments, the composition can include an amount of Compound 1, or a pharmaceutically acceptable salt or prodrug thereof, in the range of about 9000 mg to about 50 mg. In other embodiments, the composition can include an amount of Compound 1, or a pharmaceutically acceptable salt or prodrug thereof, in the range of about 5000 mg to about 150 mg. In still other embodiments, the composition can include an amount of Compound 1, or a pharmaceutically acceptable salt or prodrug thereof, in the range of about 2000 mg to about 300 mg. In yet still other embodiments, the composition can include an amount of Compound 1, or a pharmaceutically acceptable salt or prodrug thereof, in the range of about 1000 mg to about 450 mg. In an embodiment, the composition can include an amount of Compound 1, or a pharmaceutically acceptable salt or prodrug thereof, in the range of about 1000 mg to about 500 mg.

Similarly, various amounts of Compound 2, or a pharmaceutically acceptable salt or prodrug thereof, can be included in the compositions. In some embodiments, the composition can include an amount of Compound 2, or a pharmaceutically acceptable salt or prodrug thereof, in the range of about 2000 mg to about 2 mg. In other embodiments, the composition can include an amount of Compound 2, or a pharmaceutically acceptable salt or prodrug thereof, in the range of about 1600 mg to about 25 mg. In still other embodiments, the composition can include an amount of Compound 2, or a pharmaceutically acceptable salt or prodrug thereof, in the range of about 500 mg to about 50 mg. In an embodiment, the composition can include an amount of Compound 2, or a pharmaceutically acceptable salt or prodrug thereof, in the range of about 200 mg to about 100 mg.

Similarly, various amounts of Compound 3, or a pharmaceutically acceptable salt or prodrug thereof, can be included in the compositions. In some embodiments, the composition can include an amount of Compound 3, or a pharmaceutically acceptable salt or prodrug thereof, in the range of about 5000 mg to about 50 mg. In other embodiments, the composition can include an amount of Compound 3, or a pharmaceutically acceptable salt or prodrug thereof, in the range of about 2000 mg to about 150 mg. In still other embodiments, the composition can include an amount of Compound 3, or a pharmaceutically acceptable salt or prodrug thereof, in the range of about 1500 mg to about 200 mg. In yet still other embodiments, the composition can include an amount of Compound 3, or a pharmaceutically acceptable salt or prodrug thereof, in the range of about 1000 mg to about 300 mg. In an embodiment, the composition can include an amount of Compound 3, or a pharmaceutically acceptable salt or prodrug thereof, in the range of about 800 mg to about 400 mg.

A potential advantage of utilizing a combination of Compounds 1, 2, and 3, or pharmaceutically acceptable salts or prodrugs thereof, may be a reduction in the required amounts of one or more compounds that are effective in treating a disease condition disclosed herein (for example, HCV), as compared to monotherapy treatment of an otherwise comparable patient population using either Compound 1, 2 or 3, or pharmaceutically acceptable salts or prodrugs thereof, alone. In some embodiments, the amount of Compound 1, or a pharmaceutically acceptable salt or prodrug thereof, in the composition can be less compared to the amount of Compound 1 or a pharmaceutically acceptable salt or prodrug thereof, needed to achieve the same viral load reduction when administered as a monotherapy. In some embodiments, the amount of Compound 2 or a pharmaceutically acceptable salt or prodrug thereof, in the composition can be less compared to the amount of Compound 2, or a pharmaceutically acceptable salt or prodrug thereof, needed to achieve the same viral load reduction when administered as a monotherapy. In some embodiments, the amount of Compound 3 or a pharmaceutically acceptable salt or prodrug thereof, in the composition can be less compared to the amount of Compound 3, or a pharmaceutically acceptable salt or prodrug thereof, needed to achieve the same viral load reduction when administered as a monotherapy.

In an embodiment, the sum of the amount of Compound 1, or a pharmaceutically acceptable salt or prodrug thereof, and the amount of Compound 2, or a pharmaceutically acceptable salt or prodrug thereof, and the amount of Compound 3, or a pharmaceutically acceptable salt or prodrug thereof, is less than expected or predicted based on the additive combination of Compound 1, or a pharmaceutically acceptable salt or prodrug thereof, alone, Compound 2, or a pharmaceutically acceptable salt or prodrug thereof, alone, and Compound 3, or a pharmaceutically acceptable salt or prodrug thereof alone for treating the disease condition such as HCV.

Additional advantages of utilizing a combination of Compounds 1, 2, and 3, or pharmaceutically acceptable salts or prodrugs thereof, may include little to no cross resistance between Compounds 1, 2 and 3, or pharmaceutically acceptable salts or prodrugs thereof; different routes for elimination of Compounds 1, 2, and 3 or pharmaceutically acceptable salts or prodrugs thereof; little to no overlapping toxicities between Compounds 1, 2, and 3 or pharmaceutically acceptable salts or prodrugs thereof; little to no significant effects on cytochrome P450; and/or little to no pharmacokinetic interactions between Compounds 1, 2, and 3 or pharmaceutically acceptable salts or prodrugs thereof.

The percentages of Compounds 1, 2, and 3 or pharmaceutically acceptable salts or prodrugs thereof, present in the composition can also vary. For example, in some embodiments, the composition can include an amount of Compound 1, or a pharmaceutically acceptable salt or prodrug thereof, in the range of about 1% to about 98% (weight/weight) based on the sum of the amount of Compound 1, or a pharmaceutically acceptable salt or prodrug thereof, and the amount of Compounds 2 and 3, or pharmaceutically acceptable salts or prodrugs thereof, in the composition. Additional embodiments include, but are not limited to, an amount of Compound 1, or a pharmaceutically acceptable salt or prodrug thereof, in the range of about 5% to about 80%, about 10% to about 70%, about 15% to about 60%, about 20% to about 50% and about 30% to about 40% (weight/weight) based on the sum of the amount of Compound 1, or a pharmaceutically acceptable salt or prodrug thereof, and the amount of Compounds 2 and 3, or pharmaceutically acceptable salts or prodrugs thereof, in the composition.

As to Compound 2, in an embodiment, the composition can include an amount of Compound 2, or a pharmaceutically acceptable salt or prodrug thereof, in the range of about 1% to about 98% (weight/weight) based on the sum of the amount of Compound 2, or a pharmaceutically acceptable salt or prodrug thereof, and the amount of Compounds 1 and 3, or pharmaceutically acceptable salts or prodrugs thereof, in the composition. Examples of additional embodiments, include, but are not limited to, an amount of Compound 2, or a pharmaceutically acceptable salt or prodrug thereof, in the range of about 5% to about 80%, about 10% to about 70%, about 15% to about 60%, about 20% to about 50% and about 30% to about 40% (weight/weight) based on the sum of the amount of Compound 2, or a pharmaceutically acceptable salt or prodrug thereof, and the amount of Compounds 1 and 3, or pharmaceutically acceptable salts or prodrugs thereof, in the composition.

As to Compound 3, in an embodiment, the composition can include an amount of Compound 3, or a pharmaceutically acceptable salt or prodrug thereof, in the range of about 1% to about 98% (weight/weight) based on the sum of the amount of Compound 3, or a pharmaceutically acceptable salt or prodrug thereof, and the amount of Compounds 1 and 2, or pharmaceutically acceptable salts or prodrugs thereof, in the composition. Examples of additional embodiments, include, but are not limited to, an amount of Compound 3, or a pharmaceutically acceptable salt or prodrug thereof, in the range of about 5% to about 80%, about 10% to about 70%, about 15% to about 60%, about 20% to about 50% and about 30% to about 40% (weight/weight) based on the sum of the amount of Compound 3, or a pharmaceutically acceptable salt or prodrug thereof, and the amount of Compounds 1 and 2, or pharmaceutically acceptable salts or prodrugs thereof, in the composition.

Additional therapeutic agents can also be included in a composition that includes Compounds 1, 2, and 3 or pharmaceutically acceptable salts or prodrugs thereof. In some embodiments, the additional therapeutic agent can be an anti-viral agent. In an embodiment, the anti-viral agent can be a HCV anti-viral agent. A non-limiting list of examples of suitable therapeutic agents include nucleotides and nucleoside analogs (such as azidothymidine (AZT) (zidovudine), and analogs and derivatives thereof; 2′,3′-dideoxyinosine (DDI) (didanosine), and analogs and derivatives thereof; 2′,3′-dideoxycytidine (DDC) (dideoxycytidine), and analogs and derivatives thereof; 2′,3′-didehydro-2′,3′-dideoxythymidine (D4T) (stavudine), and analogs and derivatives thereof; combivir; abacavir; adefovir dipivoxil; cidofovir; ribavirin; ribavirin analogs; levovirin; viramidine; isatoribine and the like), pirfenidone or a pirfenidone analogs, tumor necrosis factor antagonists (such as etanercept, infliximab and adalimumab), thymosin-α, (Zadaxin™), an interferon receptor agonist(s), α-glucosidase inhibitors, TNF-α, antagonists, NS3 helicase inhibitors, inhibitors of NSSB polymerase (such as GS-9190, MK-3281, VCH-759 (VX-759), VCH-916, ABT-333, BMS-791325, PF-00868554, IDX-184, R1626, PSI-7851, VCH-222 (VX-222), ABT-072, and BI207127) and inhibitors of the NS5A protein (such as BMS-790052, A-831, and AZD2836), NS3 protease inhibitors (for example, (VX-950) and (SCH 503034)), toll-like receptor (TLR) modulators (such as ANA773, IMO-2125, and PF-04878691), cytochrome P450 monooxygenase inhibitors, and ribozymes such as Heptazyme™ and phosphorothioate oligonucleotides, which are complementary to HCV protein sequences and which inhibit the expression of viral core proteins.

In one embodiment, the nucleoside analog is selected from the group consisting of ribavirin, levovirin, viramidine, an L-nucleoside, and isatoribine. A preferred nucleoside analog is ribavirin.

Cytochrome P450 (CYP P450) is a very large and diverse superfamily of hemoproteins. Both exogenous and endogenous compounds are substrates for cytochrome P450 isoforms. Cytochrome P450 3A4 (CYP3A4; EC 1.14.13.97), is one of the most important enzymes involved in the metabolism of xenobiotics in the body. CYP3A4 is involved in the oxidation of the largest range of substrates of all the CYPs. Although CYP3A4 is predominantly found in the liver, it is also present in other organs and tissues of the body.

In early preclinical studies, cytochrome P450 phenotyping using chemical inhibitors suggests that multiple CYP isozymes including 3A4, 2C19, 1A2, 2D6, and 2C9 participate in the metabolism of Compound 2. Further experiments with recombinant CYPs show that only CYP3A4 metabolized Compound 2 to an extent that could influence the pharmacokinetics. Therefore, a cytochrome P450 monooxygenase inhibitor in an amount effective to inhibit metabolism of the protease inhibitor could increase the bioavailability of Compound 2 compared to administration in the absence of the CYP inhibitor.

Any CYP inhibitor that improves the pharmacokinetics of the relevant NS3 protease (e.g., Compound 2) may be used as an additional therapeutic agent in a composition or method of this invention. These CYP inhibitors include, but are not limited to, ritonavir (International Publication No. WO 94/14436), ketoconazole, troleandomycin, 4-methyl pyrazole, cyclosporin, clomethiazole, cimetidine, itraconazole, fluconazole, miconazole, fluvoxamine, fluoxetine, nefazodone, sertraline, indinavir, nelfinavir, amprenavir, fosamprenavir, saquinavir, lopinavir, delavirdine and erythromycin. A preferred CYP inhibitor is ritonavir.

Ritonavir is a potent inhibitor of CYP3A4 activity and is currently utilized at low non-therapeutic doses (e.g., about 100 mg to 200 mg twice daily) to enhance or “boost” the PK of HIV protease inhibitors (PIs), such as Compound 2 or Compound 2a.

One limitation of early interferon (IFN) therapy was rapid clearance of the protein from the blood. Chemical derivatization of IFN with polyethyleneglycol (PEG) has resulted in proteins with substantially improved pharmacokinetic properties. PEGASYS® is a conjugate of α-2a and a 40 kD branched mono-methoxy PEG and PEG-INTRON® is a conjugate of α-2b and a 12 kD mono-methoxy PEG. B. A. Luxon et al., Clin. Therapy. 2002, 24(9): 13631-1383; and A. Kozlowski and J. M Harris, J. Control. Release, 2001, 72: 217-224. However, some patients are unable or unwilling to subject themselves to interferon therapy for one or more reasons, for example, having to give themselves self-injections and/or one or more side effects related to interferon therapy.

Compounds 1, 2, and 3 or pharmaceutically acceptable salts or prodrugs thereof, can further include an additional therapeutic agent that is an interferon receptor agonist, such as a Type I interferon agonist and/or a Type II interferon agonist. In an embodiment, the Type II interferon agonist can be interferon-γ (IFN-γ). In an embodiment, the Type 1 interferon agonist can be interferon-α (IFN-α), for example, monoPEG (30 kD, linear)-ylate consensus, INFERGEN consensus IFN-α, a 40 kD branched mono-methoxy PEG conjugate of interferon α-2a and/or a 12 kD mono-methoxy PEG conjugate of interferon α-2b. In a one embodiment the Type 1 interferon agonist is a 40 kD branched mono-methoxy PEG conjugate of interferon α-2a.

As used herein, the term “interferon receptor agonist” refers to any Type I interferon receptor agonist, Type II interferon receptor agonist, or Type III interferon receptor agonist. As used herein, the term “a Type I interferon receptor agonist” refers to any naturally occurring or non-naturally occurring ligand of human Type I interferon receptor, which binds to and causes signal transduction via the receptor. Type I interferon receptor agonists include interferons, including naturally-occurring interferons, modified interferons, synthetic interferons, pegylated interferons, fusion proteins comprising an interferon and a heterologous protein, shuffled interferons; antibody specific for an interferon receptor; non-peptide chemical agonists; and the like. As used herein, the term “Type II interferon receptor agonist” refers to any naturally occurring or non-naturally occurring ligand of human Type II interferon receptor that binds to and causes signal transduction via the receptor. Type II interferon receptor agonists include native human interferon-γ, recombinant IFN-γ species, glycosylated IFN-γ species, pegylated IFN-γ species, modified or variant IFN-γ species, IFN-γ fusion proteins, antibody agonists specific for the receptor, non-peptide agonists, and the like. As used herein, the term “a Type III interferon receptor agonist” refers to any naturally occurring or non-naturally occurring ligand of humanIL-28 receptor α (“IL-28R”), the amino acid sequence of which is described by Sheppard, et al., infra., that binds to and causes signal transduction via the receptor.

In some embodiments, a composition comprising Compounds 1, 2, and 3 or pharmaceutically acceptable salts or prodrugs thereof, does not include an interferon receptor agonist.

Suitable α-glucosidase inhibitors include any of the above-described imino-sugars, including long-alkyl chain derivatives of imino sugars as disclosed in U.S. Patent Publication No. 2004/0110795; inhibitors of endoplasmic reticulum-associated α-glucosidases; inhibitors of membrane bound α-glucosidase; miglitol (Glyset®), and active derivatives, and analogs thereof; and acarbose (Precose), and active derivatives, and analogs thereof.

The compositions described herein can be administered to a human patient per se, or in compositions where they are mixed with other active ingredients, as in combination therapy, or carriers, diluents, excipients or combinations thereof, and may be formulated into preparations in solid, semi-solid, liquid or gaseous forms, such as tablets, capsules, powders, granules, ointments, solutions, suppositories, injections, inhalants and aerosols. Proper formulation is dependent upon the route of administration chosen. Techniques for formulation and administration of the compositions described herein are known to those skilled in the art. Pharmaceutically acceptable excipients are known to those skilled in the art, and are described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the compositions disclosed herein may be provided as salts with pharmaceutically compatible counterions.

In some embodiments, the compounds, or pharmaceutically acceptable salts or prodrugs thereof, (e.g., Compounds 1, 1a, 2, 2a and 3) are formulated in an aqueous buffer. Suitable aqueous buffers include, but are not limited to, acetate, succinate, citrate, and phosphate buffers varying in strengths from about 5 mM to about 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

Suitable routes of administration may, for example, include oral, rectal, topical transmucosal, or intestinal administration; parenteral delivery, including intramuscular, subcutaneous, intravenous, intramedullary injections, as well as intrathecal, direct intraventricular, intraperitoneal, intranasal, intraocular injections or as an aerosol inhalant. The compositions will generally be tailored to the specific intended route of administration. In an embodiment, the compositions described herein can be administered orally.

Subcutaneous administration can be accomplished using standard methods and devices, e.g., needle and syringe, a subcutaneous injection port delivery system, and the like. See, e.g., U.S. Pat. Nos. 3,547,119; 4,755,173; 4,531,937; 4,311,137; and 6,017,328. A combination of a subcutaneous injection port and a device for administration of a pharmaceutical composition of the embodiments to a patient through the port is referred to herein as “a subcutaneous injection port delivery system.” In many embodiments, subcutaneous administration is achieved by bolus delivery by needle and syringe.

For oral preparations, the compounds can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The compounds can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Furthermore, the compounds can be made into suppositories by mixing with a variety of bases such as emulsifying bases or water-soluble bases. The compounds of the embodiments can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the embodiments calculated in an amount sufficient to produce the desired effect in association with a pharmaceutically acceptable diluent, carrier or vehicle. The specifications for the novel unit dosage forms of the embodiments depend on the particular compound employed and the effect to be achieved, and the pharmacodynamics associated with each compound in the host.

The compositions described herein can be administered orally, parenterally or via an implanted reservoir. In an embodiment, the composition can be orally administered or administered by injection.

One may also administer the composition in a local rather than systemic manner, for example, via injection of the composition directly into the infected area, often in a depot or sustained release formulation. Furthermore, one may administer the composition in a targeted drug delivery system, for example, in a liposome coated with a tissue-specific antibody. The liposomes will be targeted to and taken up selectively by the organ.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound disclosed herein formulated in a compatible pharmaceutical carrier may also be prepared, placed in an appropriate container, and labeled for treatment of an indicated condition.

Some embodiments described herein relate to a method for ameliorating or treating a disease condition that can include administering an amount of Compound 1, or a pharmaceutically acceptable salt or prodrug thereof, an amount of Compound 2, or a pharmaceutically acceptable salt or prodrug thereof, and an amount of Compound 3, or a pharmaceutically acceptable salt or prodrug thereof wherein the disease condition can be a hepatitis C virus infection, liver fibrosis, and/or impaired liver function. In an embodiment, the prodrug of Compound 1 can be Compound 1a. In another embodiment, the salt of Compound 2 can be Compound 2a.

Various dosages forms of Compound 1, or a pharmaceutically acceptable salt or prodrug thereof, and/or Compound 2, or a pharmaceutically acceptable salt or prodrug thereof, and/or Compound 3, or a pharmaceutically acceptable salt or prodrug thereof, can be used to ameliorate and/or treat a disease condition. In some instances, Compounds 1, 2, and 3 or pharmaceutically acceptable salts or prodrugs thereof, can be present in the same dosage form such as the compositions described herein. In other instances, Compounds 1, 2 and 3, or pharmaceutically acceptable salts or prodrugs thereof, can be administered as separate dosage forms. For example, Compound 1, or a pharmaceutically acceptable salt or prodrug thereof, can be administered in one tablet, Compound 2, or a pharmaceutically acceptable salt or prodrug thereof, can be administered in a second tablet, and Compound 3, or a pharmaceutically acceptable salt or prodrug thereof, can be administered in a third tablet. When Compounds 1, 2, and 3 or pharmaceutically acceptable salts or prodrugs thereof, are contained in separate dosage forms, the dosage forms can be the same (e.g., as both pills) or different (e.g., two compounds can be formulated in a pill and the other compound can be formulated as an injectable).

Administration of Compound 1, or a pharmaceutically acceptable salt or prodrug thereof, Compound 2, or a pharmaceutically acceptable salt or prodrug thereof, and Compound 3, or a pharmaceutically acceptable salt or prodrug thereof can vary. When Compounds 1, 2, and 3, or pharmaceutically acceptable salts or prodrugs thereof, are contained in separate dosage forms, the dosage forms can be administered simultaneously or sequentially. In some embodiments, the dosage form that contains Compound 1, or a pharmaceutically acceptable salt or prodrug thereof, can be administered before, after, in-between, concurrently or sequentially with Compounds 2 and 3, or pharmaceutically acceptable salts or prodrugs thereof. In some embodiments, the dosage form that contains Compound 2, or a pharmaceutically acceptable salt or prodrug thereof, can be administered before, after, in-between, concurrently or sequentially with Compounds 1 and 3, or pharmaceutically acceptable salts or prodrugs thereof. In some embodiments, the dosage form that contains Compound 3, or a pharmaceutically acceptable salt or prodrug thereof, can be administered before, after, in-between, concurrently or sequentially with Compounds 1 and 2, or pharmaceutically acceptable salts or prodrugs thereof.

In some embodiments, Compounds 1, 2, and 3, or pharmaceutically acceptable salts or prodrugs thereof, can be administered concurrently. As used, the term “concurrently” means effective concentrations of all three compounds are present in a subject. When being administered concurrently, Compounds 1, 2, and 3, or pharmaceutically acceptable salts or prodrugs thereof, can be administered in the same dosage form or separate dosage forms. In other embodiments, Compounds 1, 2, and 3, or pharmaceutically acceptable salts or prodrugs thereof, can be administered sequentially. As used herein, the term “sequentially” means administering one compound for a first time period, then administering a second compound for a second time period, and then administering a third compound for a third period, in which the first, second, and third time periods do not overlap.

Additional therapeutic agents can also be administered to the subject having the disease condition. A non-limiting list of additional therapeutic agents includes those previously described herein. When one or more additional therapeutic agents are utilized, the additional agent(s) can be administered in the same dosage form as Compound 1, or a pharmaceutically acceptable salt or prodrug thereof, and/or Compound 2, or a pharmaceutically acceptable salt or prodrug thereof, and/or Compound 3, or a pharmaceutically acceptable salt or prodrug thereof. For example, the additional therapeutic agent(s) can be included in a composition that includes Compound 1, or a pharmaceutically acceptable salt or prodrug thereof, without Compounds 2 or 3, or pharmaceutically acceptable salts or prodrugs thereof; or a composition that includes Compound 2, or a pharmaceutically acceptable salt or prodrug thereof, without Compounds 1 or 3, or pharmaceutically acceptable salts or prodrugs thereof; or a composition that includes Compound 3, or a pharmaceutically acceptable salt or prodrug thereof, without Compounds 1 or 2, or pharmaceutically acceptable salts or prodrugs thereof. In some embodiments, the additional therapeutic agent(s) can be included in a composition that includes any two of the three compounds of Compounds 1, 2, or 3; or the additional therapeutic agent(s) can be included in a composition described herein that includes Compounds 1, 2, and 3 or pharmaceutically acceptable salts or prodrugs thereof.

Alternatively, the additional therapeutic agent(s) can be administered in one or more separate dosage forms. If administered as one or more separate dosage forms, each dosage form with one or more additional therapeutic agents can be the same as the dosage form containing Compound 1, or a pharmaceutically acceptable salt or prodrug thereof, the dosage form containing Compound 2, or a pharmaceutically acceptable salt or prodrug thereof, and/or the dosage form containing Compound 3, or a pharmaceutically acceptable salt or prodrug thereof or different from the dosage form containing Compound 1, or a pharmaceutically acceptable salt or prodrug thereof, the dosage form containing Compound 2, or a pharmaceutically acceptable salt or prodrug thereof, and/or the dosage form containing Compound 3, or a pharmaceutically acceptable salt or prodrug thereof.

When one or more additional therapeutic agents are in one or more separate dosage forms, the dosage forms with one or more additional therapeutic agents can be administered before, after, in-between, concurrently or sequentially with Compound 1, or a pharmaceutically acceptable salt or prodrug thereof, Compound 2, or a pharmaceutically acceptable salt or prodrug thereof, and/or Compound 3, or a pharmaceutically acceptable salt or prodrug thereof. In some embodiments, the additional therapeutic agent can be ribavirin. In another embodiment, the additional therapeutic agent can be ritonavir. In some embodiments, Compounds 1, 2, and 3 can be administered with additional therapeutic agents that are ribavirin and ritonavir.

In some embodiments, the additional therapeutic agent can be an interferon receptor agonist, for example, a Type I interferon receptor agonist and/or a Type II interferon receptor agonist. In an embodiment, the Type II interferon agonist can be interferon-γ (IFN-γ). In an embodiment, the Type 1 interferon agonist can be interferon-α (IFN-α). In some embodiments, the Type I interferon agonist can be selected from monoPEG (30 kD, linear)-ylate consensus, INFERGEN consensus IFN-α, a 40 kD branched mono-methoxy PEG conjugate of interferon α-2a and a 12 kD mono-methoxy PEG conjugate of interferon α-2b. In an embodiment, the interferon receptor agonist can be a Type 1 interferon receptor agonist, such as a pegylated Type 1 interferon receptor agonist. In another embodiment, Compounds 1, 2, and 3 can be administered without one or more additional therapeutic agents such as an interferon receptor agonist and/or ribavirin.

One or more additional therapeutic agents can be administered prior to administration of Compound 1, or a pharmaceutically acceptable salt or prodrug thereof, Compound 2, or a pharmaceutically acceptable salt or prodrug thereof, or Compound 3, or a pharmaceutically acceptable salt or prodrug thereof. In one embodiment, one or more additional therapeutic agents can be administered prior to a treatment regimen with Compounds 1, 2, and 3, or pharmaceutically acceptable salts or prodrugs thereof. In another embodiment, the lead-in period to a treatment regimen is fourteen days. In another embodiment, the additional therapeutic agent used in the lead-in period can be ribavirin.

Whether a subject method is effective in treating an HCV infection can be determined in various ways, for example, by a reduction in viral load, a reduction in time to seroconversion (virus undetectable in patient serum), an increase in the rate of sustained viral response to therapy, a reduction of morbidity or mortality in clinical outcomes, or other indicator of disease response. Thus, whether a subject method is effective in treating an HCV infection can be determined by measuring viral load, or by measuring a parameter associated with HCV infection, including, but not limited to, liver fibrosis, elevations in serum transaminase levels, and necroinflammatory activity in the liver.

In some embodiments, administration and/or use of Compounds 1, 2, and 3 or pharmaceutically acceptable salts or prodrugs thereof, in combination can reduce the viral load more than the viral load reduction achieved by administration of any two of the three compounds of Compounds 1, 2, and 3, or pharmaceutically acceptable salts or prodrugs thereof, at substantially the same amount. For example, the combination of Compounds 1, 2, and 3 or pharmaceutically acceptable salts or prodrugs thereof, may reduce the viral load by at least about 10%, at least about 15%, at least about 20%, at least about 25%, at least about 30%, at least about 40%, at least about 50%, at least about 60%, at least about 70%, at least about 80%, or at least about 90%, or more, as compared to the reduction of HCV viral load achieved by substantially the same amount of any two of the three compounds of Compound 1, 2, and 3, or pharmaceutically acceptable salts or prodrugs thereof, administered as a combination therapy.

In some embodiments, administration and/or use of Compounds 1, 2, and 3 or pharmaceutically acceptable salts or prodrugs thereof, in combination with one or more additional therapeutic agents can reduce the viral load more than the viral load reduction achieved by administration of any two of the three compounds of Compound 1, 2, and 3, or pharmaceutically acceptable salts or prodrugs thereof, with one or more additional therapeutic agents, at substantially the same amount. For example, the combination of Compounds 1, 2, and 3 or pharmaceutically acceptable salts or prodrugs thereof, along with one or more additional therapeutic agents may reduce the viral load by at least about 10%, at least about 15%, at least about 20%, at least about 25%, at least about 30%, at least about 40%, at least about 50%, at least about 60%, at least about 70%, at least about 80%, or at least about 90%, or more, as compared to the reduction of HCV viral load achieved by substantially the same amount of any two of the three compounds of Compound 1, 2, and 3, or pharmaceutically acceptable salts or prodrugs thereof, along with one or more additional therapeutic agents, administered as a combination therapy.

In some embodiments, an amount of Compound 1, or a pharmaceutically acceptable salt or prodrug thereof, an amount of Compound 2, or a pharmaceutically acceptable salt or prodrug thereof, and an amount of Compound 3, or a pharmaceutically acceptable salt or prodrug thereof, is a synergistic amount. As used herein, a “synergistic combination” or a “synergistic amount” is a combined dosage that is more effective in the therapeutic or prophylactic treatment of an HCV infection than the incremental improvement in treatment outcome that could be predicted or expected from a merely additive combination of (i) the therapeutic or prophylactic benefit of Compound 1, or a pharmaceutically acceptable salt or prodrug thereof, (ii) the therapeutic or prophylactic benefit of Compound 2, or a pharmaceutically acceptable salt or prodrug thereof, and (iii) the therapeutic or prophylactic benefit of Compound 3, or a pharmaceutically acceptable salt or prodrug thereof, when administered at the same dosage as a combination therapy.

The term “synergistic combination” or a “synergistic amount” may also be used to refer to a combined dosage that is more effective in the therapeutic or prophylactic treatment of an HCV infection than could be predicted or expected, based on the rule of mixtures, from a combination of (i) the therapeutic or prophylactic benefit of Compound 1, or a pharmaceutically acceptable salt or prodrug thereof; (ii) the therapeutic or prophylactic benefit of Compound 2, or a pharmaceutically acceptable salt or prodrug thereof; and (iii) the therapeutic or prophylactic benefit of Compound 3, or a pharmaceutically acceptable salt or prodrug thereof. Accordingly, in some embodiments, the combination of Compounds 1, 2, and 3, or pharmaceutically acceptable salts or prodrugs thereof, may reduce the viral load by at least about 10%, at least about 15%, at least about 20%, at least about 25%, at least about 30%, at least about 40%, at least about 50%, at least about 60%, at least about 70%, at least about 80%, or at least about 90%, or more, as compared to the reduction in HCV viral load predicted or expected from the rule of mixtures or additive combination of the viral load reductions from administration of Compound 1, or a pharmaceutically acceptable salt or prodrug thereof, Compound 2, or a pharmaceutically acceptable salt or prodrug thereof, and Compound 3, or a pharmaceutically acceptable salt or prodrug thereof. In some embodiments, the foregoing levels of viral load reduction are averages based on a population of subjects. HCV viral load and viral load reduction can be determined by methods known in the art. For example, HCV viral load may be determined by measuring HCV RNA levels using a suitable assay such as a reverse transcriptase PCR assay. In one embodiment, the assay is the COBAS® AmpilPrep/COBAS® Taqman® HCV Test RUO (“Research Use Only”).

The combination of Compounds 1, 2, and 3, or pharmaceutically acceptable salts or prodrugs thereof, may shorten the time period it takes a subject to achieve a sustained viral response to therapy. For example, the combination of Compounds 1, 2, and 3, or pharmaceutically acceptable salts or prodrugs thereof, may shorten the time period it takes a subject to achieve a sustained viral response to therapy compared to the time period it takes the subject to achieve a sustained viral response being administered substantially the same amount of any two of the three compounds of Compounds 1, 2, and 3, or pharmaceutically acceptable salts or prodrugs thereof.

In an embodiment, the combination of Compounds 1, 2, and 3, or pharmaceutically acceptable salts or prodrugs thereof, may shorten the time period it takes a subject to achieve a sustained viral response to therapy by at least about 10%, at least about 15%, at least about 20%, at least about 25%, at least about 30%, at least about 40%, at least about 50%, at least about 60%, at least about 70%, at least about 80%, or at least about 90%, or more, as compared to that expected based on the rule of mixtures or additive combination expected or predicted from the time period it takes the subject to achieve a sustained viral response to therapy being administered substantially the same amount of Compounds 1, 2, and 3, or pharmaceutically acceptable salts or prodrugs thereof. In some embodiments, the time periods for achieving a sustained viral response are averages based on a population of subjects.

As noted above, whether a subject method is effective in treating an HCV infection can be determined by measuring a parameter associated with HCV infection, such as liver fibrosis. Methods of determining the extent of liver fibrosis are known to those skilled in the art. In some embodiments, the level of a serum marker of liver fibrosis indicates the degree of liver fibrosis.

As a non-limiting example, levels of serum alanine aminotransferase (ALT) are measured, using standard assays. In general, an ALT level of less than about 45 international units is considered normal. In some embodiments, the combination of Compounds 1, 2, and 3, or pharmaceutically acceptable salts or prodrugs thereof, reduces a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in a subject being administered substantially the same amount of any two of the three compounds of Compounds 1, 2, and 3, or pharmaceutically acceptable salts or prodrugs thereof.

In other embodiments, the combination of Compounds 1, 2, and 3, or pharmaceutically acceptable salts or prodrugs thereof, reduces a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, as compared to that expected based on the rule of mixtures or the additive combination of the levels of reduction of a serum level of a marker of liver fibrosis using substantially the same amount of Compounds 1, 2, and 3, or pharmaceutically acceptable salts or prodrugs thereof. In some embodiments, the reduction of serum levels of a marker of liver fibrosis are averages based on a population of subjects.

A subject being treated for a disease condition can experience resistance to one or more of the therapeutic agents (for example, Compound 1, or a pharmaceutically acceptable salt or prodrug thereof, and/or Compound 2, or a pharmaceutically acceptable salt or prodrug thereof, and/or Compound 3, or a pharmaceutically acceptable salt or prodrug thereof). The term “resistance” as used herein refers to a subject displaying a delayed, lessened and/or absent response to the therapeutic agent(s). For example, the viral load of a subject with HCV who has become resistant to an anti-viral or combination thereof may be reduced to a lesser degree compared to the amount in viral load reduction exhibited by the subject before becoming resistant to the anti-viral or combination thereof and/or the determined normal mean viral load reduction. In some embodiments, the level of resistance of the disease condition to therapy can be decreased compared to the level of resistance measured in a subject being administered substantially the same amount of any two of the three compounds of Compounds 1, 2, and 3, or pharmaceutically acceptable salts or prodrugs thereof. In other embodiments, the combination of Compounds 1, 2, and 3, or pharmaceutically acceptable salts or prodrugs thereof, reduces the level of resistance of the disease condition to therapy by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, as compared to that expected based on the rule of mixtures or additive combination of the levels of resistance using substantially the same amount of Compounds 1, 2, and 3, or pharmaceutically acceptable salts or prodrugs thereof. In some embodiments, the levels of resistance are averages based on a population of subjects.

Some subjects being treated for HCV who develop or have resistance to one or more therapies experience a viral load rebound. The term “viral load rebound” as used herein refers to a sustained ≧0.5 log IU/ml increase of viral load above nadir before the end of treatment, where nadir is a ≧0.5 log IU/ml decrease from baseline. In some embodiments, administration of Compound 1, or pharmaceutically acceptable salts or prodrugs thereof, Compound 2, or pharmaceutically acceptable salts or prodrugs thereof, and Compound 3, or pharmaceutically acceptable salts or prodrugs thereof, results in less subjects experiencing a viral load rebound as compared to monotherapy involving one of Compounds 1, 2, or 3, or a pharmaceutically acceptable salt or prodrug thereof, or administration of any two of the three compounds of Compounds 1, 2, and 3, or pharmaceutically acceptable salts or prodrugs thereof. In some embodiments, the administration of Compounds 1, 2, and 3, or pharmaceutically acceptable salts or prodrugs thereof, results in at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, reduction in number of subjects experiencing a viral load rebound as compared to monotherapy involving one of Compounds, 1, 2, or 3, or a pharmaceutically acceptable salt or prodrug thereof, or administration of any two of the three compounds of Compounds 1, 2, and 3, or pharmaceutically acceptable salts or prodrugs thereof. In some embodiments, the administration of Compounds 1, 2, and 3, or pharmaceutically acceptable salts or prodrugs thereof, results in less than about 75%, less than about 50%, less than about 40%, less than about 30%, less than about 20%, less than about 10%, or less than about 5% of the patient population who experiences a viral load rebound. In other embodiments, the combination of Compounds 1, 2, and 3, or pharmaceutically acceptable salts or prodrugs thereof, reduces the percentage of the patient population who experiences a viral load rebound by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, as compared to that expected based on the rule of mixtures.

Some subjects being treated for HCV who develop or have resistance for one or more therapies are or become non-responders. The term “non-responder” as used herein refers to a viral load decrease <0.5 log IU/ml during treatment. In some embodiments, administration of Compound 1, or a pharmaceutically acceptable salt or prodrug thereof, Compound 2, or a pharmaceutically acceptable salt or prodrug thereof, and Compound 3, or a pharmaceutically acceptable salt or prodrug thereof, results in less subjects who are non-responders as compared to monotherapy involving one of Compounds 1, 2, or 3, or pharmaceutically acceptable salts or prodrugs thereof, or administration of any two of the three compounds of Compound 1, 2, and 3, or pharmaceutically acceptable salts or prodrugs thereof. In some embodiments, the administration of Compounds 1, 2, and 3, or pharmaceutically acceptable salts or prodrugs thereof, results in at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, reduction in number of patients who are non-responders as compared to monotherapy involving one of Compounds, 1, 2, or 3, or pharmaceutically acceptable salts or prodrugs thereof, or administration of any two of the three compounds of Compounds 1, 2, and 3, or pharmaceutically acceptable salts or prodrugs thereof. In some embodiments, the administration of Compounds 1, 2, and 3, or pharmaceutically acceptable salts or prodrugs thereof, results in less than about 75%, less than about 50%, less than about 40%, less than about 30%, less than about 20%, less than about 10%, or less than about 5% of the patient population who are non-responders. In other embodiments, the combination of Compounds 1, 2, and 3, or pharmaceutically acceptable salts or prodrugs thereof, reduces the percentage of the patient population who are non-responders by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, as compared to that expected based on the rule of mixtures.

In addition or in the alternative, in some embodiments, the onset of resistance of the disease condition to therapy can be delayed compared to when the onset of resistance occurs in a subject being administered substantially the same amount of any two of the three compounds of Compounds 1, 2, and 3 or pharmaceutically acceptable salts or prodrugs thereof. In some embodiments, the onset of resistance of the disease condition to therapy can be delayed compared to when the onset of resistance occurs in a subject being administered substantially the same amount of two of the three compounds of Compounds 1, 2, and 3, or pharmaceutically acceptable salts or prodrugs thereof. As used herein, the phrase “onset of resistance” is the point in time when the subject shows resistance to one or more therapeutic compounds. In an embodiment, the disease can be HCV. In some embodiments, the combination of Compounds 1, 2, and 3, or pharmaceutically acceptable salts or prodrugs thereof, may be a synergistic combination in that the onset of resistance may be delayed by at least about 10%, at least about 15%, at least about 20%, at least about 25%, at least about 30%, at least about 40%, at least about 50%, at least about 60%, at least about 70%, at least about 80%, or at least about 90%, or more, as compared to when the onset of resistance is predicted or expected based on the rule of mixtures or the additive combination of substantially the same amounts of Compounds 1, 2, and 3, or pharmaceutically acceptable salts or prodrugs thereof. In some embodiments, the time of the onset of resistance is an average based on a population of subjects.

Often one or more side effects are experienced by subjects being treated with therapeutic agents such as anti-viral compounds. In some instances, the side effects may be to such a degree that treatment with the agent may not be feasible or recommended such that treatment is not an option for some subjects or treatment has to be stopped. By lessening or decreasing the number and/or severity of the side effects, subject compliance with the treatment may be increased. In some embodiments, the number of side effects associated with administration of Compound 1, or a pharmaceutically salt or prodrug thereof; Compound 2, or a pharmaceutically acceptable salt or prodrug thereof; and Compound 3, or a pharmaceutically acceptable salt or prodrug thereof can be less than the number of side effects exhibited by the subject being administered substantially the same amount of Compound 1, 2, or 3, or a pharmaceutically acceptable salt or prodrug thereof, as the only active agent. In some embodiments, the number of side effects associated with administration of Compound 1, or a pharmaceutically salt or prodrug thereof, Compound 2, or a pharmaceutically acceptable salt or prodrug thereof, and Compound 3, or a pharmaceutically acceptable salt or prodrug thereof can be less than the number of side effects exhibited by the subject being administered substantially the same amount of any two of the three compounds of Compounds 1, 2, and 3, or pharmaceutically acceptable salts or prodrugs thereof. In other embodiments, the subject being administered a combination of Compounds 1, 2, and 3 or pharmaceutically acceptable salts or prodrugs thereof, may exhibit less side effects than predicted or expected based on the rule of mixtures or the additive combination of side effects experienced by a subject being administered substantially the same amounts of Compounds 1, 2, and 3, or pharmaceutically acceptable salts or prodrugs thereof, by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%. In some embodiments, the number of side effects is an average based on a population of subjects.

As previously stated, compliance by subjects to the anti-viral treatment may also be increased by decreasing the severity of one or more side effects that is associated with monotherapy with the active compounds. In some embodiments, the severity of a side effect associated with the combination of Compound 1, or a pharmaceutically acceptable salt or prodrug thereof, Compound 2, or a pharmaceutically acceptable salt or prodrug thereof, and Compound 3, or a pharmaceutically acceptable salt or prodrug thereof is decreased compared to the severity of the side effect experienced by the subject being administered Compound 1, 2, or 3, or a pharmaceutically acceptable salt or prodrug thereof, as a monotherapy. In some embodiments, the severity of a side effect associated with the combination of Compound 1, or a pharmaceutically acceptable salt or prodrug thereof, Compound 2, or a pharmaceutically acceptable salt or prodrug thereof, and Compound 3, or a pharmaceutically acceptable salt or prodrug thereof may be decreased by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, as compared to the severity of the side effect predicted or expected based on the rule of mixtures or the additive combination of the severities of the side effect associated with substantially the same amount of Compound 1, or a pharmaceutically acceptable salt or prodrug thereof, substantially the same amount of Compound 2, or a pharmaceutically acceptable salt or prodrug thereof, and substantially the same amount of Compound 3, or a pharmaceutically acceptable salt or prodrug thereof. In some embodiments, the severity of a side effect is an average based on a population of subjects.

As will be readily apparent to one skilled in the art, the useful in vivo dosage to be administered and the particular mode of administration will vary depending upon the age, weight, the severity of the affliction, and mammalian species treated, the particular compounds employed, and the specific use for which these compounds are employed. (See e.g., Fingl et al. 1975, in “The Pharmacological Basis of Therapeutics”, which is hereby incorporated herein by reference in its entirety, with particular reference to Ch. 1, p. 1). The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

Although the exact dosage will be determined on a drug-by-drug basis, in most cases, some generalizations regarding the dosage can be made. The dosage may be a single one or a series of two or more given in the course of one or more days, as is needed by the subject. In some embodiments, the compounds will be administered for a period of continuous therapy, for example for a week or more, or for months or years.

In instances where human dosages for compounds have been established for at least some condition, those same dosages, or dosages that are between about 0.1% and 500%, more preferably between about 25% and 250% of the established human dosage will be used. Where no human dosage is established, as will be the case for newly-discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the above-stated, preferred dosage range in order to effectively and aggressively treat particularly aggressive diseases or infections.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response was not adequate (precluding toxicity). The magnitude of an administrated dose in the management of the disorder of interest will vary with the severity of the condition to be treated and the route of administration. The severity of the condition may, for example, be evaluated, in part, by standard prognostic evaluation methods. Further, the dose and perhaps dose frequency will also vary according to the age, body weight, and response of the individual patient. A program comparable to that discussed above may be used in veterinary medicine.

In non-human animal studies, applications of potential products are commenced at higher dosage levels, with dosage being decreased until the desired effect is no longer achieved or adverse side effects disappear. The dosage may range broadly, depending upon the desired effects and the therapeutic indication. Alternatively dosages may be based and calculated upon the surface area of the patient, as understood by those of skill in the art.

Compounds disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, or monkeys, may be determined using known methods. The efficacy of a particular compound may be established using several recognized methods, such as in vitro methods, animal models, or human clinical trials. Similarly, acceptable animal models may be used to establish efficacy of chemicals to treat such conditions. When selecting a model to determine efficacy, the skilled artisan can be guided by the state of the art to choose an appropriate model, dose, and route of administration, and regime. Of course, human clinical trials can also be used to determine the efficacy of a compound or composition in humans.

Any of the compositions and methods described herein can be administered to individuals who have been diagnosed with an HCV infection. Any of the compositions and methods described herein can be administered to individuals who have failed previous treatment for HCV infection (“treatment failure patients,” including non-responders and relapsers).

Individuals who have been clinically diagnosed as infected with HCV are of particular interest in many embodiments. Individuals who are infected with HCV are identified as having HCV RNA in their blood, and/or having anti-HCV antibody in their serum. Such individuals include anti-HCV ELISA-positive individuals, and individuals with a positive recombinant immunoblot assay (RIBA). Such individuals may also, but need not, have elevated serum ALT levels.

Individuals who are clinically diagnosed as infected with HCV include naïve individuals (e.g., individuals not previously treated for HCV, particularly those who have not previously received IFN-α-based and/or ribavirin-based therapy) and individuals who have failed prior treatment for HCV (“treatment failure” patients). Treatment failure patients include non-responders (i.e., individuals in whom the HCV titer was not significantly or sufficiently reduced by a previous treatment for HCV, e.g., a previous IFN-α monotherapy, a previous IFN-α and ribavirin combination therapy, or a previous pegylated IFN-α and ribavirin combination therapy); and relapsers (i.e., individuals who were previously treated for HCV, e.g., who received a previous IFN-α monotherapy, a previous IFN-α and ribavirin combination therapy, or a previous pegylated IFN-α and ribavirin combination therapy, whose HCV titer decreased, and subsequently increased).

In an embodiment, HCV-positive individuals have an HCV titer of at least about 10⁵, at least about 5×10⁵, or at least about 10⁶, or at least about 2×10⁶, genome copies of HCV per milliliter of serum. The patient may be infected with any HCV genotype (genotype 1, including 1a and 1b, 2, 3, 4, 6, etc. and subtypes (e.g., 2a, 2b, 3a, etc.)), particularly difficult to treat genotypes such as HCV genotype 1, and particular HCV subtypes and quasispecies. In one embodiment, the patient is infected with HCV genotype 1b.

In some embodiments, the HCV-positive individuals (as described above) are those who exhibit severe fibrosis or early cirrhosis (non-decompensated, Child's-Pugh class A or less), or more advanced cirrhosis (decompensated, Child's-Pugh class B or C) due to chronic HCV infection and who are viremic despite prior anti-viral treatment with IFN-α-based therapies or who cannot tolerate IFN-α-based therapies, or who have a contraindication to such therapies. In an embodiment, HCV-positive individuals with stage 3 or 4 liver fibrosis according to the METAVIR scoring system are suitable for treatment with the compositions and methods described herein. In other embodiments, individuals suitable for treatment with the compositions and methods described herein are patients with decompensated cirrhosis with clinical manifestations, including patients with far-advanced liver cirrhosis, including those awaiting liver transplantation. In still other embodiments, individuals suitable for treatment with the compositions and methods described herein include patients with milder degrees of fibrosis including those with early fibrosis (stages 1 and 2 in the METAVIR, Ludwig, and Scheuer scoring systems; or stages 1, 2, or 3 in the Ishak scoring system).

### EXAMPLES

Embodiments are disclosed in further detail in the following examples, which are not in any way intended to limit the scope of the claims.

### Example 1

### Treatment Protocols

A dose ranging study of Compounds 1a, 2a, and 3, plus ribavirin (RBV or R) and ritonavir (RTV or r) was conducted in adult patients with chronic hepatitis C genotype. Approximately 110 treatment-naïve males and females ≧18 years of age (inclusive) with genotype 1 HCV infection who had not previously been treated with an interferon or investigational HCV therapeutic agent were enrolled.

Five groups, A, B, C, D, and E of subjects were studied. Blood samples (5 ml) were collected to determine plasma concentrations of the compounds at time points to determine pharmacokinetics parameters, including Tₘₐₓ, Cₘₐₓ, T₁₁₂, and AUC. SVR4 was measured as an efficacy parameter.

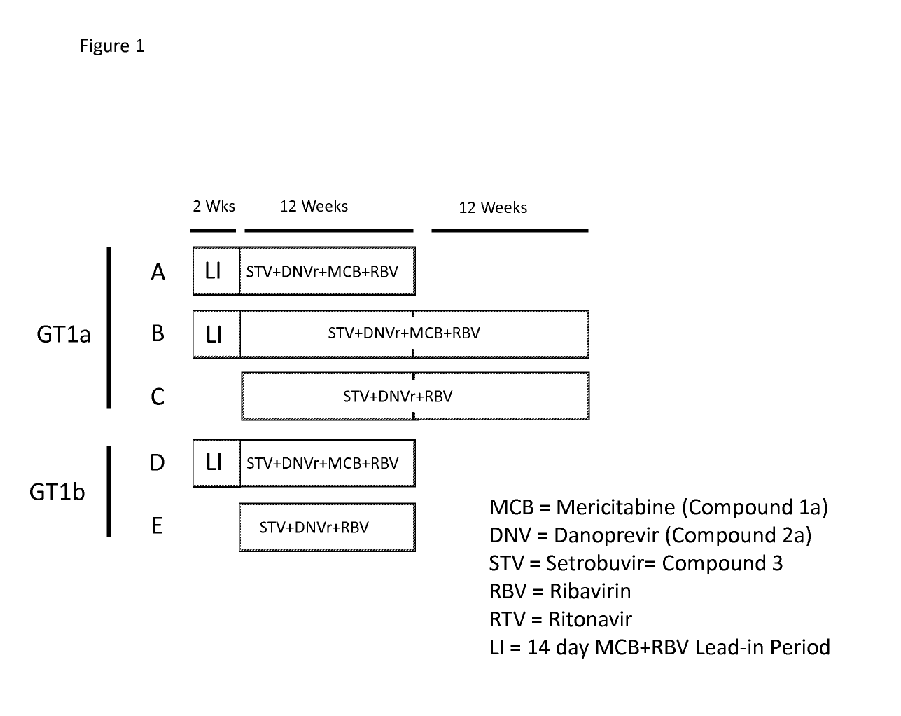

The specifics of the groups are shown in Table 1 below and pictorially in FIG. 1. All groups include approximately 22 subjects. Groups A, B, and C consist of treatment-naïve HCV genotype 1a subjects. Groups D and E consist of treatment-naïve HCV genotype 1b subjects. Groups A, B, and D have a lead in period, wherein Compound 1a and ribavirin are dosed from weeks 1 to 2 according to Table 1.

TABLE 1

| Group | Compound | Amount | Frequency | Regime |
|---|---|---|---|---|
| A | 1a | 1000 mg | bid | Weeks 1 to 14 |
| | 2a | 100 mg | bid | Weeks 3 to 14 |
| | 3 | 800 mg Day 1 | bid | Weeks 3 to 14 |
| | | then 400 mg | | |
| | RBV | 1000 mg (<75 kg) | day | Weeks 1 to 14 |
| | | or | | |
| | | 1200 mg (≧75 kg) | | |
| | RTV | 100 mg | bid | Weeks 3 to 14 |
| B | 1a | 1000 mg | bid | Weeks 1 to 26 |
| | 2a | 100 mg | bid | Weeks 3 to 26 |
| | 3 | 800 mg Day 1 | bid | Weeks 3 to 26 |
| | | then 400 mg | | |
| | RBV | 1000 mg (<75 kg) | day | Weeks 1 to 26 |
| | | or | | |
| | | 1200 mg (≧75 kg) | | |
| | RTV | 100 mg | bid | Weeks 3 to 26 |
| C | 1a | 0 mg | NA | |
| | 2a | 100 mg | bid | Weeks 1 to 24 |
| | 3 | 800 mg Day 1 | bid | Weeks 1 to 24 |
| | | then 400 mg | | |
| | RBV | 1000 mg (<75 kg) | day | Weeks 1 to 24 |
| | | or | | |
| | | 1200 mg (≧75 kg) | | |
| | RTV | 100 mg | bid | Weeks 1 to 24 |
| D | 1a | 1000 mg | bid | Weeks 1 to 14 |
| | 2a | 100 mg | bid | Weeks 3 to 14 |
| | 3 | 800 mg Day 1 | bid | Weeks 3 to 14 |
| | | then 400 mg | | |
| | RBV | 1000 mg (<75 kg) | day | Weeks 1 to 14 |
| | | or | | |
| | | 1200 mg (≧75 kg) | | |
| | RTV | 100 mg | bid | Weeks 3 to 14 |
| E | 1a | 0 mg | NA | |
| | 2a | 100 mg | bid | Weeks 1 to 12 |
| | 3 | 800 mg Day 1 | bid | Weeks 1 to 12 |
| | | then 400 mg | | |
| | RBV | 1000 mg (<75 kg) | day | Weeks 1 to 12 |
| | | or | | |
| | | 1200 mg (≧75 kg) | | |
| | RTV | 100 mg | bid | Weeks 1 to 12 |

bid—twice daily
NA—not applicable
1000 mg (<75 kg)—Administering 1000 mg of RBV to a patient weighing 75 kgs or less

Plasma concentrations of the compounds were measured by validated liquid chromatography/tandem mass spectrometry (LC-MS/MS) methods. The pharmacokinetic parameters for each compound were estimated using standard non-compartmental methods using WinNonlin (Version 5.2, Pharsight Co.) using standard methods.

### HCV RNA Viral Load Determination and Viral Resistance Assessment

Blood samples for HCV RNA assessments (anti-viral activity ±resistance) were collected throughout the treatment and follow-up period.

Approximately 10 mL of blood was used for both the HCV RNA viral load determination and the viral resistance assessment. The HCV RNA levels were determined by COBAS® AmpilPrep/COBAS® Taqman® HCV Test RUO (“Research-Use-Only”). This is a real-time PCR method. HCV and RNA measurements were taken at designated time points. Mean and individual plots of viral load data (absolute and change from baseline) were provided from each group. A listing of individual change from baseline was determined. Summaries of HCV RNA measurements at each nominal time point were provided by treatment group.

Selected blood samples collected for viral load determinations were utilized for phenotypic and sequence analyses to monitor for development of resistance to compounds 1a, 2a, and 3 in subjects, who can experience either viral load rebound or non-response while on treatment with compounds 1a, 2a, and 3.

Population sequencing of the complete coding sequence of the HCV NSSB polymerase and/or NS3/4A of all baseline samples was performed using standard sequencing technology. For subjects experiencing viral load rebound, attempts were made to determine the population NSSB coding sequence at (a) baseline and (b) at the first sample after viral load rebound. Amino acid substitutions in the samples after viral load rebound were determined as compared to the respective baseline sequence for each selected subject. Secondary analyses include sequencing the entire HCV genome, sequencing of samples derived from subjects having a virological response, and determining sequences of minority quasispecies. Phenotype studies to monitor for resistance to Compounds 1a, 2a, and 3 of the samples outlined in (a) and (b) were performed, and include analysis of samples derived from subjects having a virological response. Assessment of cross resistance to other HCV inhibitors and sequences analyses were performed on selected samples and may require amplification and subcloning of sequences from the HCV genome.

As provided below in Table 2, GT1b patients treated with the combination of Compounds 1a, 2a, and 3 plus ribavirin and ritonavir showed a decrease in viral load or SVR4 (96% vs. 77%) compared to patients treated with only Compounds 2a and 3, plus ribavirin and ritonavir. For GT1a patients, the overall SVR4 rate was 74% for patients receiving 26 weeks of therapy compared with 43% for those receiving 14 weeks of therapy. Two groups (A and C) were discontinued due to meeting pre-defined futility rules.

TABLE 2

| | GT1a | | GT1b | |
|---|---|---|---|---|
| | Group A | Groups A + B | Group D | Group E |
| | 14 wks | 26 wks | 14 wks | 12 wks |
| | (n = 7) | (n = 47) | (n = 23) | (n = 22) |
| RVR % | 96 | | 100 | 100 |
| EOT % | 100 | 89 | 100 | 100 |
| SVR4 % | 43 | 74 | 96 | 77 |

n = number of patients
RVR = rapid viral response (<25 IU/ml after 4 weeks of therapy)
EOT = end of treatment

Subjects treated with the combination of compounds 1a, 2a, and 3, without pegylated interferon, experience a median reduction in viral levels in all regimens and patient populations tested.

### Safety and Tolerability

The safety and tolerability results for the combination treatment using Compounds 1a, 2a, and 3, plus ribavirin and ritonavir, and without pegylated interferon demonstrate that the combination was well tolerated, and no major safety concerns were identified. Most adverse events were mild and moderate.

It will be understood by those of skill in the art that numerous and various modifications can be made without departing from the spirit of the present application. Therefore, it should be clearly understood that the forms of the present application are illustrative only and not intended to limit the scope of the present application.

1. A composition comprising
(i) a first compound, or a pharmaceutically acceptable salt thereof, wherein the first compound is 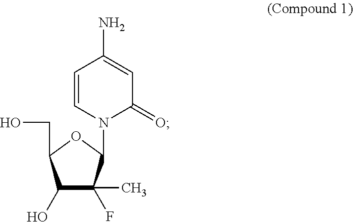
or a prodrug of the first compound, wherein the prodrug is 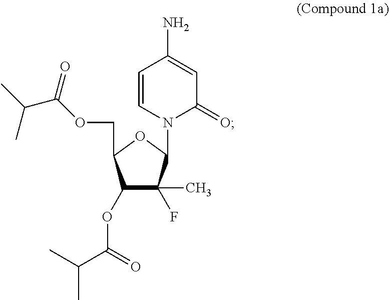
(ii) a second compound, or a pharmaceutically acceptable salt thereof, wherein the second compound is 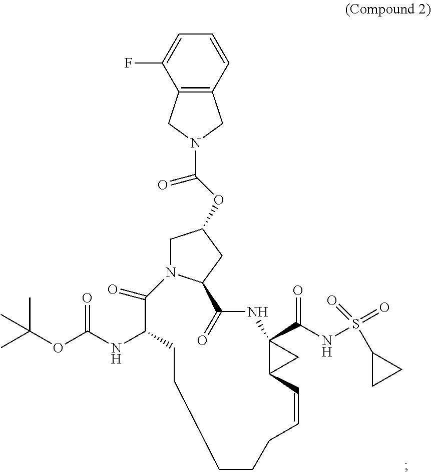 [SMILES: CC(C)(C)OC(=O)N[C@H]1CCCCC/C=C\[C@@H]2C[C@@]2(C(=O)NS(=O)(=O)C2CC2)NC(=O)[C@@H]2C[C@@H](OC(=O)N3Cc4cccc(F)c4C3)CN2C1=O]
(iii) a third compound, or a pharmaceutically acceptable salt thereof, wherein the third compound is 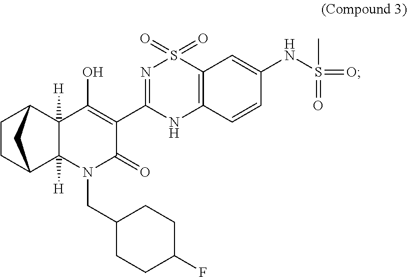
and wherein the composition further comprises ribavirin and ritonavir.

8. The composition of claim 1, wherein the composition does not comprise an interferon.

10. The composition of claim 1, wherein the salt of the second compound is a sodium salt (Compound 2a).

12. The composition claim 1, further comprising a pharmaceutically acceptable excipient, diluent or carrier.

13. A combination of
(i) a composition comprising a therapeutically effective amount of a first compound, or a pharmaceutically acceptable salt thereof, wherein the first compound is 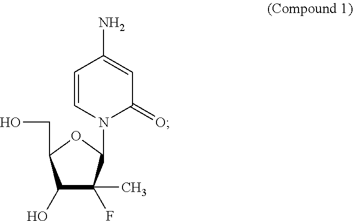
or a composition comprising a therapeutically effective amount of a prodrug of compound 1, wherein the prodrug is 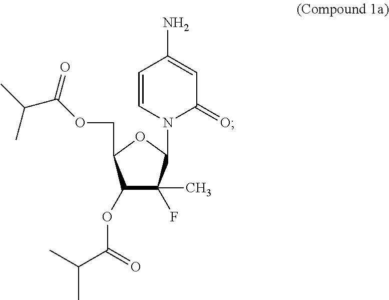
(ii) a composition comprising a therapeutically effective amount of a second compound, or a pharmaceutically acceptable salt thereof, wherein the second compound is 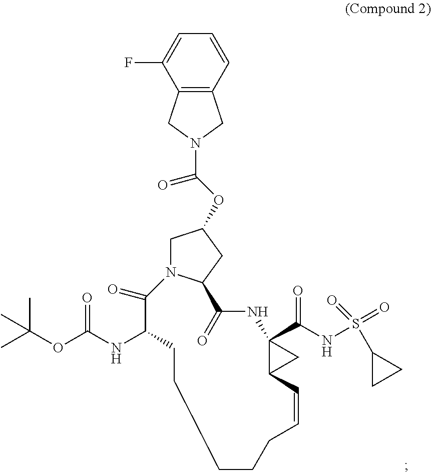 [SMILES: CC(C)(C)OC(=O)N[C@H]1CCCCC/C=C\[C@@H]2C[C@@]2(C(=O)NS(=O)(=O)C2CC2)NC(=O)[C@@H]2C[C@@H](OC(=O)N3Cc4cccc(F)c4C3)CN2C1=O]
and (iii) a composition comprising a therapeutically effective amount of a third compound, or a pharmaceutically acceptable salt thereof, wherein the third compound is 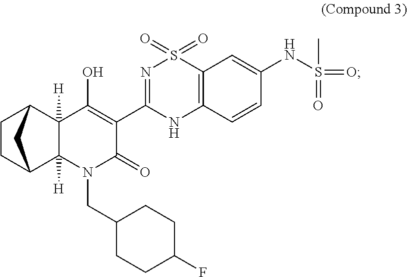
wherein the combination further comprises ribavirin and ritonavir for use in treating a subject suffering from a disease condition, wherein the disease condition is selected from the group consisting of a hepatitis C virus infection, liver fibrosis, and impaired liver function.

14. The combination of claim 13 for use in treating a subject suffering from a genotype 1b hepatitis C virus infection.

15. The combination of claim 13 wherein the compositions are administered separately in one or more unit dosage forms.

16. The combination of claim 13 wherein the compositions comprising Compound 1 and Compound 2, Compound 1 and Compound 3, or Compound 2 and Compound 3, or pharmaceutically acceptable salts thereof, are administered together in one or more unit dosage forms.

17. The combination of claim 16, wherein Compound 1 and Compound 3, or pharmaceutically acceptable salts thereof, are administered together.

18. The combination of claim 17 wherein the compositions are administered in a pack or dispenser device.

19. The combination of claim 18 wherein the pack or dispenser device is a blister pack.

20. A method for ameliorating or treating a disease condition in a patient population comprising administering a therapeutically effective amount of
(i) a first compound, or a pharmaceutically acceptable salt thereof, wherein the first compound is 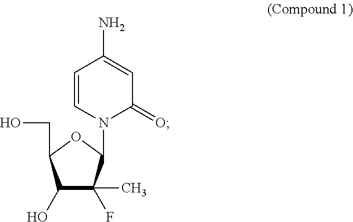
or a prodrug of the first compound, wherein the prodrug is 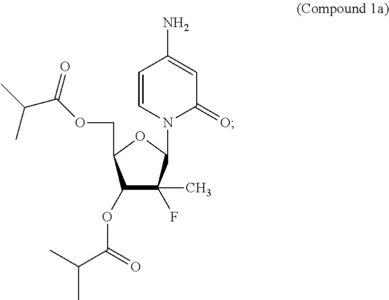
(ii) a therapeutically effective amount of a second compound, or a pharmaceutically acceptable salt thereof, wherein the second compound is 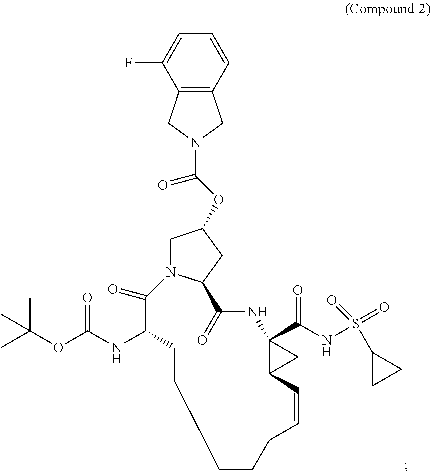 [SMILES: CC(C)(C)OC(=O)N[C@H]1CCCCC/C=C\[C@@H]2C[C@@]2(C(=O)NS(=O)(=O)C2CC2)NC(=O)[C@@H]2C[C@@H](OC(=O)N3Cc4cccc(F)c4C3)CN2C1=O]
(iii) a therapeutically effective amount of a third compound, or a pharmaceutically acceptable salt thereof, wherein the third compound is 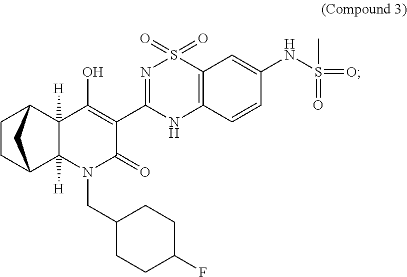
and (iv) ritonavir and ribavirin to a subject suffering from the disease condition, wherein the disease condition is selected from a hepatitis C virus infection, liver fibrosis, and impaired liver function.

30. The method of claim 20, wherein the salt of the second compound is a sodium salt (Compound 2a).

32. The method of claim 20, wherein Compounds 1, 2, and 3, or pharmaceutically acceptable salts thereof, are administered concurrently.

33. The method of claim 20, wherein Compounds 1, 2, and 3, or pharmaceutically acceptable salts thereof, are together in one dosage form.

34. The method of claim 20, wherein Compounds 1, 2, and 3, or pharmaceutically acceptable salts thereof, are in separate dosage forms.

35. The method of claim 20, wherein Compound 1 and Compound 2, Compound 1 and Compound 3, or Compound 2 and Compound 3, or pharmaceutically acceptable salts thereof, are administered together as a therapeutic agent in the same dosage form.

36. The method of claim 20, wherein Compound 1 and Compound 3, or pharmaceutically acceptable salts thereof, are administered together as a therapeutic agent in the same dosage form.

37. The method of claim 20 for treating a subject suffering from a genotype 1b hepatitis C virus infection.
